(19) United States
(12) Patent Application Publication
(10) Pub. No.: US 2024/0148808 A1
(43) Pub. Date: May 9, 2024

(54) RECOMBINANT VSV FOR THE TREATMENT OF BLADDER CANCER

(71) Applicant: SOCIETE DE COMMERCIALISATION DES PRODUITS DE LA RECHERCHE APPLIQUEE SOCPRA SCIENCES SANTE ET HUMAINE, Sherbrooke (CA)

(72) Inventor: Lee-Hwa TAI, Sherbrooke (CA)

(21) Appl. No.: 18/567,179

(22) PCT Filed: Jun. 10, 2022
(86) PCT No.: PCT/CA2022/050925

§ 371 Date: Dec. 5, 2023

Related U.S. Application Data
(60) Provisional application No. 63/209,681, filed on Jun. 11, 2021.

(51) Int. Cl. A61K 35/766; A61K 9/00; A61K 35/74; A61K 38/19; A61K 38/21; A61P 35/00

(52) U.S. Cl. CPC A61K 35/766; A61K 9/0019; A61K 35/74; A61K 38/193; A61K 38/212; A61P 35/00

(57) ABSTRACT

It is provided a composition comprising (i) VSVd51-hGM-CSF construct as depicted in SEQ ID NO: 1, (ii) a functional equivalent thereof, or (iii) a nucleotide sequence having at least 70% identity with SEQ ID NO: 1; and a carrier, for treating a solid cancer such as e.g. bladder cancer.

### CROSS-REFERENCE TO RELATED APPLICATIONS

The present application claims benefit of U.S. Provisional Application No. 63/209,861 filed Jun. 11, 2021 the content of which is herewith incorporated in its entirety.

### TECHNICAL FIELD

It is provided the use of oncolytic VSVd51-hGM-CSF for the treatment of a solid cancer.

### BACKGROUND

Bladder cancer (BC) is consistently among the six most prevalent cancers in men with approximately 500,000 new cases diagnosed annually worldwide. BC poses significant clinical problems and is the most expensive solid cancer to treat due to the requirement for specialized monitoring equipment and highly trained personnel. Most BC cases are non-muscle invasive (NMIBC), but they are associated with high recurrence rates and a significant risk of progression to muscle invasive disease. Transurethral resection (TUR) along with intravesical chemo- or immunotherapies have been shown to decrease the recurrence and/or progression rates of NMIBC. However, despite these front-line therapies, 50-80% of patients will develop recurrence at 5 years, of which up to 25% will evolve into muscle invasive BC (MIBC). For high-risk NMIBC, the use of intravesical Bacillus Calmette-Guérin (BCG) has shown significant benefits in reducing recurrence and progression compared to TUR alone. Unfortunately, up to 84% of patients are unable to complete the 3 year BCG regimen due to treatment inefficacy and local and/or systemic toxic effects. Cystectomy remains the standard of treatment for high-risk patients who have failed BCG therapy. Patients who undergo cystectomy before their bladder cancer progresses to muscle invasive disease, have shown good disease free survival. However, cystectomy poses a significantly diminished quality of life.

Recent studies have shown that BCG and interferon (IFNα) combination therapy may be useful as salvage regimen in BCG failures. However, various tumors acquire defects in their ability to respond to IFNs as they evolve and many aggressive BC cell lines are highly resistant to IFN treatment. IFN-resistance confers a growth advantage for cancer cells over normal tissues, but simultaneously compromises their antiviral response. To exploit this vulnerability of aggressive BC cells, Zhang et al. (2010, Int J Cancer, 127: 830-838) used an oncolytic rhabdovirus—Vesicular Stomatitis Virus (VSV) that possesses the ability to selectively infect, replicate in and kill IFN-resistant BC cells, but is strongly suppressed in IFN-responsive normal tissues. The tumor specificity of wild-type VSV has been further enhanced in an attenuated strain (VSVd51) with a point mutation in the matrix protein, which has a defect in its ability to short-circuit the antiviral activity of IFNs, and induces an enhanced protective response in normal tissues, while maintaining its oncolytic ability. Importantly, the lack of pre-existing neutralizing antibodies in human populations, a major hurdle that impedes the in vivo delivery of many other oncolytic viruses (OVs, e.g. adeno-, herpes, vaccinia, measles virus), warrants the development of oncolytic rhabdoviruses for clinical applications.

It is thus highly desirable to be provided with alternative bladder-sparing therapies that are effective and less toxic for high-risk BC to prevent both recurrence and progression.

### SUMMARY

It is provided a composition comprising (i) VSVd51-hGM-CSF construct as depicted in SEQ ID NO: 1, (ii) a functional equivalent thereof, or (iii) a nucleotide sequence having at least 70% identity with SEQ ID NO: 1; and a carrier.

In an embodiment, the composition described herein is for treating a solid cancer.

In another embodiment, the solid cancer is bladder cancer, pancreatic cancer, breast cancer, colorectal cancer, ovarian cancer, or melanoma.

In a particular embodiment, the solid cancer is bladder cancer.

In a further embodiment, the composition is formulated for an administration selected from parenteral, intravenous, oral, subcutaneous, intra-arterial, intracranial, intrathecal, intraperitoneal, topical, intranasal and intramuscular.

It is also provided a method of treating a solid cancer comprising administering to a patient in need thereof a composition comprising a vesicular stomatitis virus (VSV) and a growth factor, wherein preferably, the VSV is VSVd51, the growth factor is a human growth factor, and the human growth factor is granulocyte macrophage-colony stimulating factor (GM-CSF).

In an embodiment, the composition comprises VSVd51-hGM-CSF construct as depicted in SEQ ID NO: 1.

It is provided that the composition is used as a monotherapy or in combination with an immunomodulator and/or Bacillus Calmette-Guérin (BCG).

In an embodiment, the immunomodulator is a type I interferon.

In an embodiment, the immunomodulator is an interferon alpha (IFNα).

In another embodiment, the composition increases expression of at least one of CD80, CD86, HLA-DR and PD-L1 in said patient.

In a further embodiment, the composition increases ATP levels in said patient.

In a supplemental embodiment, the composition increases gene expression of at least one of CCL4, CCL5, CXCL9, CXCL10, CXCL11, IFNγ, IL6, IRF-1, CSF-2, TNFα, CSF-2, TAP1 and TAP2 compared to untreated controls.

It is also provided the use of a composition as described herein for treating a solid cancer in a patient in need thereof.

### BRIEF DESCRIPTION OF THE DRAWINGS

Reference will now be made to the accompanying drawings.

FIG. 1 illustrates the characterization of a VSVd51 oncolytic virus encoding human GM-CSF, showing in (A) a schematic of the VSVd51-hGM-CSF genome; (B) cytotoxicity of VSVd51 and VSVd51-hGM-CSF assessed in UM-UC-3 and 5637 BC cells at the indicated MOIs 48 hours after infection using Alamar Blue; (C) viral titres produced from of VSVd51 and VSVd51-hGM-CSF infected BC cells; and (D) the amount of hGM-CSF secreted from infected cells was examined 6 hours after infection by ELISA and reported as ng/ml of GM-CSF per cell, wherein n=3 for technical replicates, ***, P<0.001; ****, P<0.0001.

FIG. 2 illustrates the GM-CSF transgene in VSVd51 inducing immunogenic cell death associated gene signature in the MB49 mouse bladder cancer cell line following infection, showing in (A) Western blot analysis of HMGB1 from cell-free supernatants; (B) luminometry measurement of ATP from cell-free supernatants; (C) measurement of cell surface calreticulin of the mouse MB49 BC cell line infected with VSVd51 and VSVd51-mGM-CSF at indicated MOI and following indicated time points; and (D) fold change in gene expression of MB49 cells following infection with VSVd51 and VSVd51-mGM-CSF at 10 MOI for 24 h, wherein quantitative PCR was performed using mRNA pooled from 3 independent experiments, all data are representative of at least three similar experiments where n=3 for technical replicates, *, P<0.05; **, P<0.01; ***, P<0.001; ****, P<0.0001; (n.s., no significance).

FIG. 3 illustrates the enhanced immune cell activation following VSVd51-mGM-CSF treatment in the syngeneic C57BI/6-MB49 mouse model, showing in (A) timeline of in vivo C57BI/6-MB49 experiment, where B6 mice bladders were instilled with 1×10⁶ MB49 cells and two days later, each group of mice received via intravesical instillation 50 μl of 5×10⁸ PFU of VSVd51 or VSVd51-mGM-CSF or vehicle for control groups; innate immune cells in (B, C) were assessed via peripheral blood at day 4 post tumor implantation; adaptive and regulatory immune cells (D-G) were assessed peripherally or in the bladder on day 8 post tumor implantation; immune cell suspensions from the peripheral blood (B-D) or dissociated bladders (E) of mice following indicated treatments were stained with (B, E) NK cell markers (NK1.1⁺, CD3⁻, CD69⁺, Granzyme B⁺, IFNγ⁺), (C) DC markers (CD11c⁺, CD80⁺, CD86⁺), (D, E) T cell markers (CD3⁺, CD8⁺, Granzyme B⁺, IFNγ⁺), (F) T regulatory cells (CD3⁺, CD4⁺, CD25⁺, Foxp3⁺), (G) myeloid derived suppressor cells (CD11b⁺, Ly6G⁺, ARG1⁺) and analyzed by flow cytometry, wherein all data are representative of at least three similar experiments where n=5-8 mice/treatment, *, P<0.05; **, P<0.01; ***, P<0.001; (n.s., no significance).

FIG. 4 illustrates diminished bladder tumor burden and improved survival in the C57BI/6-MB49 model following VSVd51-mGM-CSF treatment is dependent on both immune effector and regulatory cells, showing in (A) timeline of in vivo C57BI/6-MB49 experiment. B6 mice bladders instilled with 1×10⁶ MB49 cells and two days later, each group of mice received via intravesical instillation 50 μl of 5×10⁸ PFU of VSVd51 or VSVd51-mGM-CSF or vehicle for control groups; in (B) tumor volume assessment at days 2 and 6 following indicated treatments was performed via small animal ultrasound; (C) Kaplan-Meier survival analysis of C57BI/6 mice bearing MB49 bladder tumors and treated with indicated therapies; (D) timeline of immune cell depletion in the C57BI/6-MB49 in vivo model, where one day before virus treatment, NK cells, CD8⁺ T cells and NK+CD8⁺ T cells were depleted using antibodies to GM1, CD8, GM1+CD8, and Ly6G and continued every 3 days for a total of 6 doses, and on days 2 and 3 following tumor implantation, all mice received VSVd51-mGM-CSF; and (E, F) Kaplan-Meier survival analysis of C57BI/6 mice bearing MB49 bladder tumors and receiving VSVd51-mGM-CSF and indicated antibody depletion or vehicle control, wherein n=10-12 mice/group. *, P<0.05; **, P<0.01; (n.s., no significance), log-rank test.

FIG. 5 illustrates enhanced immunogenic cell death and activation of innate and adaptive immune cells following exposure to VSVd51-hGM-CSF in human BC spheroids, showing in (A) Western blot analysis of HMGB1 (V:VSVd51; G:VSVd51-hGM-CSF) and (B) luminometry measurement of ATP from cell-free supernatants of human BC 5637 and UM-UC-3 spheroids infected with VSVd51 and VSVd51-mGM-CSF at indicated MOI and following indicated time points; in (C) polarization of purified human monocytes in the presence of CM from human BC 5637 spheroids infected with VSVd51 or VSVd51-hGM-CSF at indicated MOI and time points; and in (D) migration assay of human CD3⁻/CD56⁺ NK cells and CD3⁺/CD8⁺ T cells following exposure to CM from human BC 5637 spheroids infected with VSVd51 or VSVd51-hGM-CSF at indicated MOI and time points, wherein all data are representative of at least three similar experiments where n=3 for technical replicates. *, P<0.05; **, P<0.01; ***, P<0.001; (n.s., no significance).

FIG. 6 illustrates VSVd51-hGM-CSF enhances immune signature, biomarkers of ICD and autologous immune cell activation in human BC patient derived organoids. ICD and immune activation of BC patient derived organoids from patient 34 and 38 as measured through (A) fold change in gene expression following infection with VSVd51-hGM-CSF following 18 h and 10 MOI; showing in (B) Western blot analysis of HMGB1 (V:VSVd51; G:VSVd51-hGM-CSF); in (C) luminometry measurement of ATP; (D) functionality of autologous human CD8⁺ T and NK cells following co-culture with autologous treated-cDC and (E) activation of autologous human cDCs in the presence of CM from infected human BC patient derived organoids, wherein all organoids were infected with VSVd51 or VSVd51-hGM-CSF at indicated MOI and time points, data are pooled from technical replicates, n=3, *, P<0.05; **, P<0.01; (n.s., no significance).

### DETAILED DESCRIPTION

In accordance with the present description, there is provided the use of oncolytic VSVd51-hGM-CSF for the treatment of bladder cancer.

It is provided administering a virus (vesicular stomatitis virus, VSV) that is not a common human pathogen to reduce the viability of bladder cancer cells, while leaving normal cells largely unharmed. The encompassed virus has been engineered to contain a special human growth factor (granulocyte macrophage-colony stimulating factor, GM-CSF) that will stimulate an immune response by attracting and promoting the development of antigen presenting cells and effector immune cells. The immune response will help with the local removal of bladder cancer cells as well cancer cells that may have spread to regional lymph nodes or other organs (metastases).

Given the urgent medical need for the development of additional and less toxic bladder-sparing therapies for BC patients failing frontline treatments, a novel VSVd51 containing human GM-CSF (VSVd51-hGM-CSF) is provided. Using both the human and existing mouse variant (VSVd51-mGM-CSF), their ability to treat BC was evaluated. BC cell lines were assessed for susceptibility to viral lysis and expression of ICD markers and immune gene signatures.

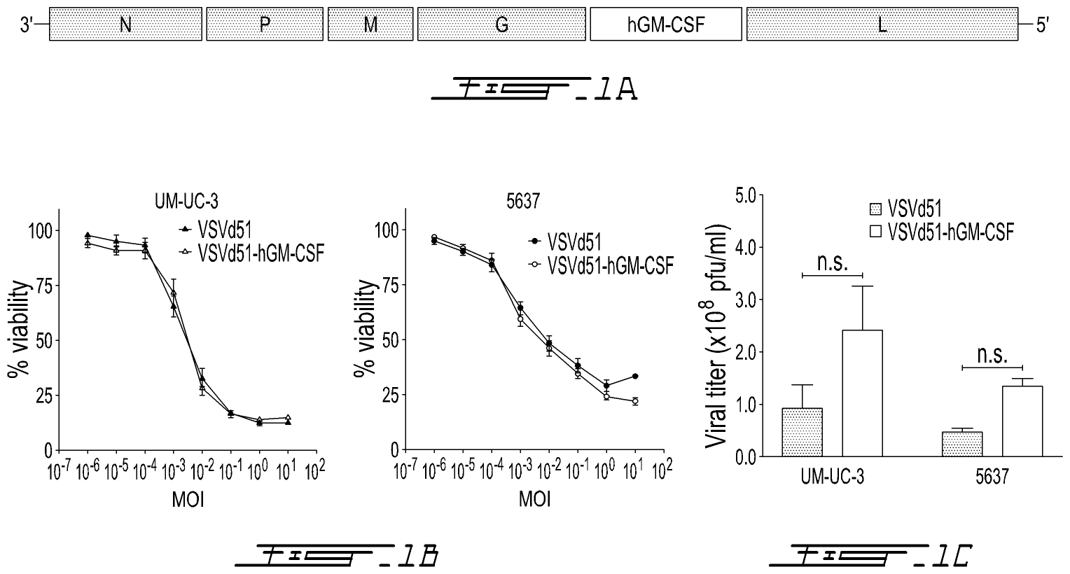

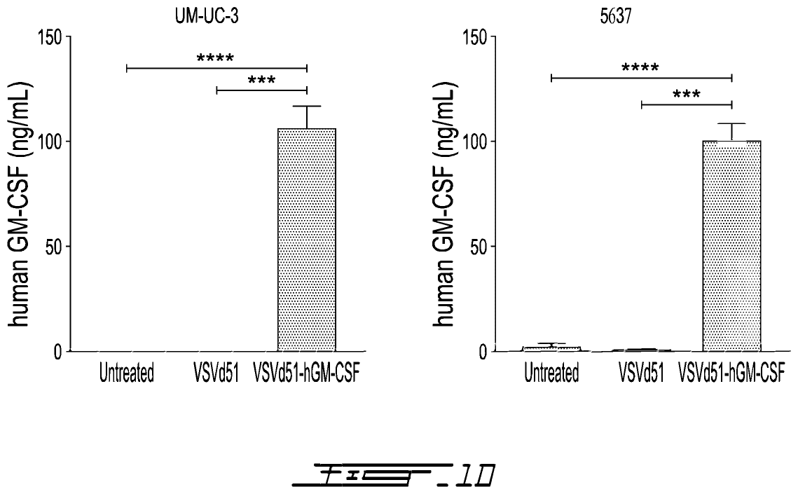

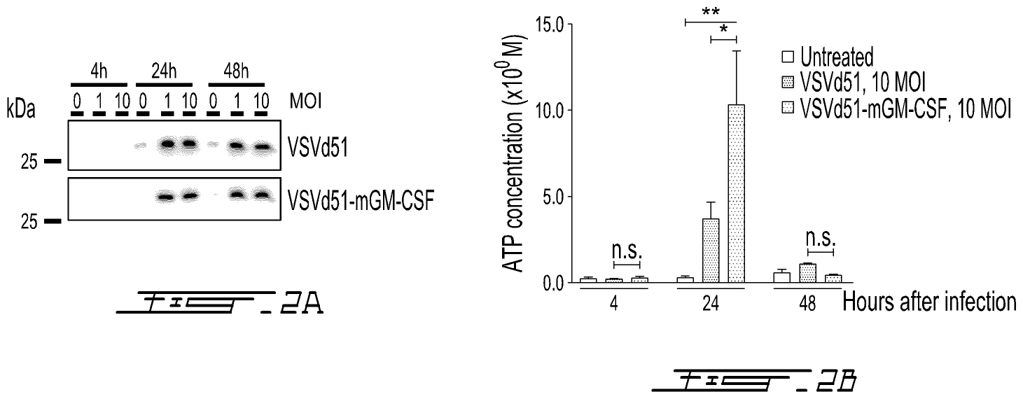

Due to the therapeutic potential of GM-CSF, a human GM-CSF transgene was incorporated into the backbone of the oncolytic VSVd51 variant to create VSVd51-hGM-CSF (FIG. 1A) (SEQ ID NO: 1). This replication competent OV could infect human BC cell lines with an efficiency comparable to parental VSVd51, and expression of GM-CSF did not negatively affect viral replication (FIGS. 1B and C). Human GM-CSF was quantified in the culture media of 5637 and UM-UC-3 cell lines infected with VSVd51-hGM-CSF (FIG. 1D). The mouse variant, VSVd51-mGM-CSF was also able to infect and replicate in a mouse MB49 bladder cancer cell line. Therefore, VSVd51-hGM-CSF could successfully infect human tumor cells, the virus could replicate and GM-CSF was secreted, resulting in a functional VSVd51-hGM-CSF.

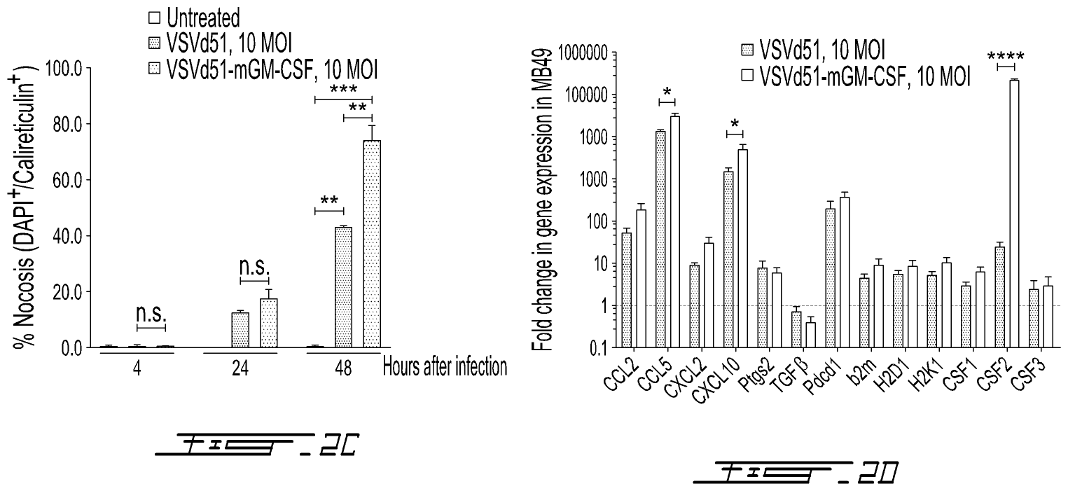

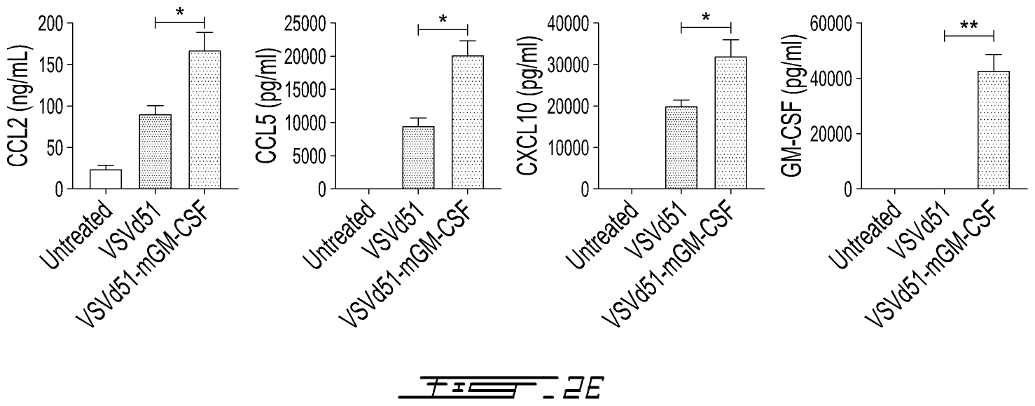

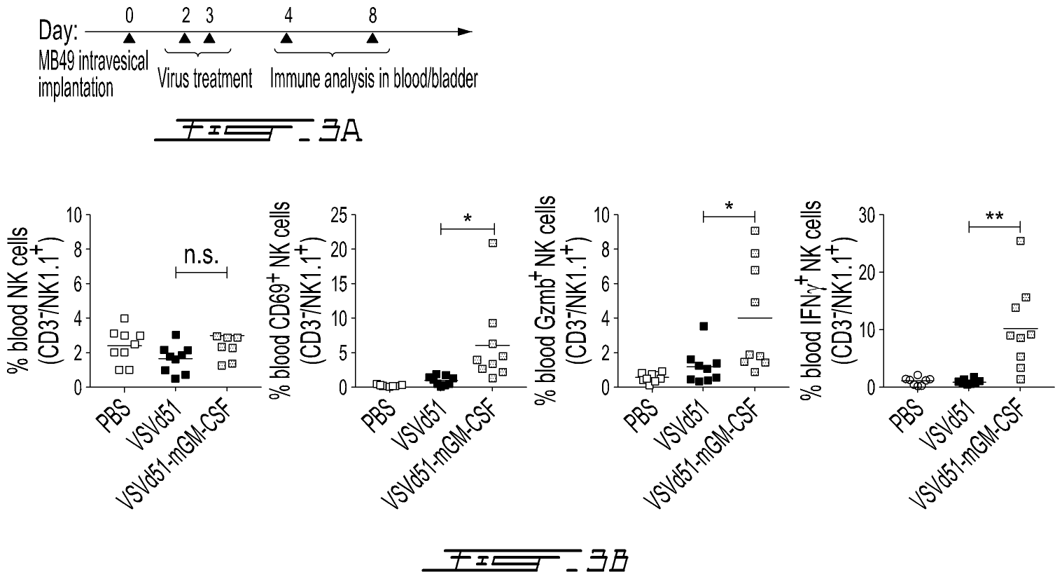

There is strong evidence to suggest that the immune system plays a critical role in determining the outcome of VSV therapy. Given the importance of the mode of tumor cell death in initiating anti-tumor immune response, ICD was assessed following infection of the mouse MB49 cell line with VSVd51 or VSVd51-mGM-CSF. High mobility group box 1 (HMGB1) protein was first measured (FIG. 2A) and adenosine triphosphate (ATP) (FIG. 2B) in the supernatant of infected cells at various time points post-infection. ATP release was increased following 24 h of infection and similar HMGB1 levels by the VSVd51-mGM-CSF compared to controls. Another feature of necrosis is the presence of cell surface externalized calreticulin. Following virus infection, an increase in the percentage of necrotic (calreticulin⁺/DAPI⁺) cells was observed in VSVd51-mGM-CSF treated cells at 48 h post-infection (FIG. 2C). Together, the presence of these heightened danger associated molecular patterns (DAMPs) suggest a greater induction of ICD by VSVd51-mGM-CSF.

A panel of genes related to pro-inflammatory, anti-inflammatory, antigen presentation and immune differentiation markers was examined by qPCR. Twenty-four hours following infection with the viruses, a general upregulation of genes related to immune cell recruitment and activation was detected in MB49 cells. Notably, mouse CCL2, CCL5, CXCL2, CXCL10 and GM-CSF transcripts showed an increase in expression in MB49 cells following VSVd51-mGM-CSF infection compared to VSVd51 and non-infected controls (FIG. 2D). MHC-I related genes such as β2m and H2-D also showed an upregulated pattern of expression upon infection by VSVd51-mGM-CSF, although the results were not significant. These data suggest enhanced immunogenic gene expression in MB49 cells following VSVd51-mGM-CSF induced ICD.

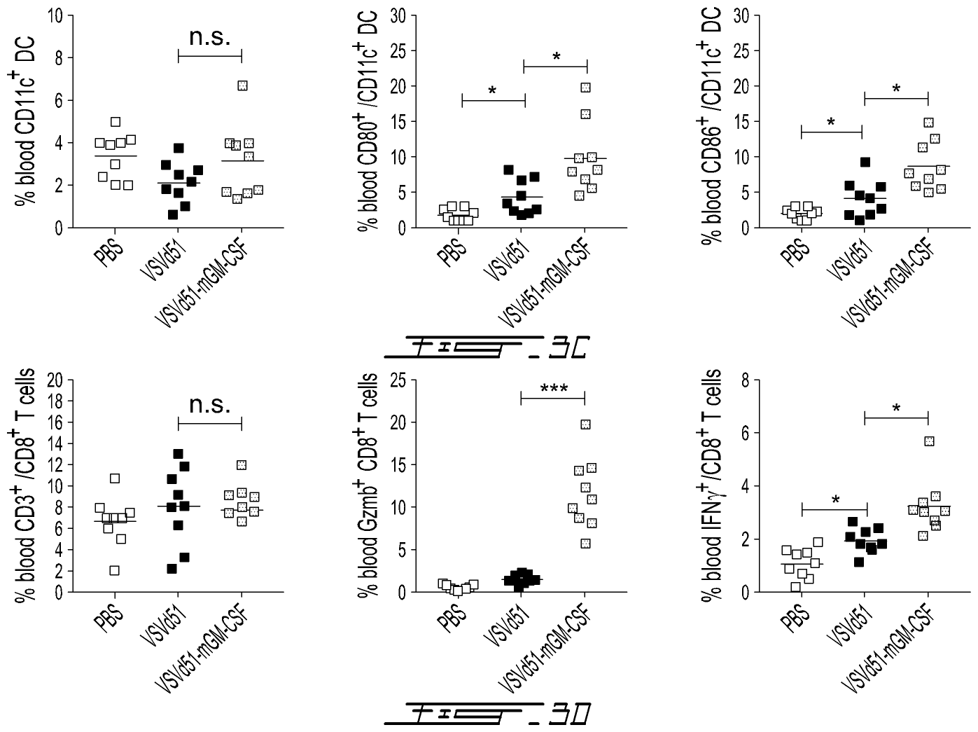

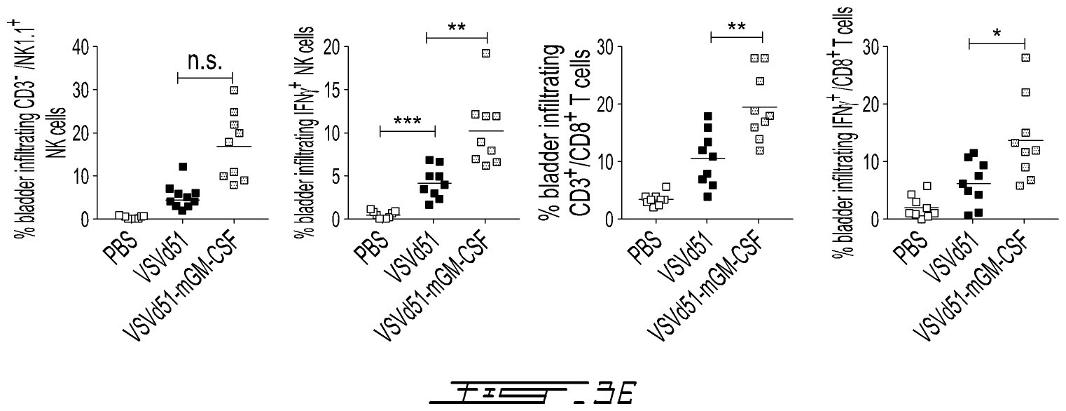

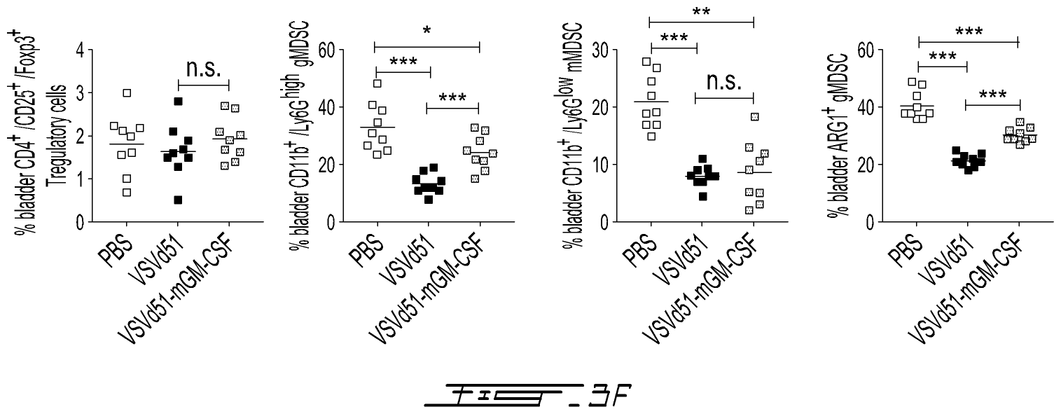

To determine if the observed in vitro ICD and immune gene signatures in mouse BC cells, translate to improved immune function in vivo, VSVd51 and VSVd51-mGM-CSF were compared in the treatment of C57BI/6 mice bearing orthotopic MB49 tumors (FIG. 3A, timeline). MB49 is one of the most-used murine bladder carcinoma cell lines and shares pivotal immunological and cell surface tumor characteristics with aggressive human BC. At early and late time points following intravesical VSVd51-mGM-CSF treatment, a significant increased proportions of CD69⁺ (early lymphocyte activation), IFNγ+(cytokine production) and Granzyme B⁺ (cytotoxicity) peripheral NK cells were observed compared to treatment with the parental virus or PBS (FIG. 3B). Similar results were observed with CD11c⁺ conventional dendritic cells (cDC) in terms of their activation status (CD80⁺ and CD86⁺) (FIG. 3C). Analysis of peripheral CD3⁺/CD8⁺ T cells showed increased IFNγ, and Granzyme B in VSVd51-mGM-CSF treated mice over controls (FIG. 3D). However, the proportion of NK, DC and CD8⁺ T cell numbers in the periphery remained constant across treatments (FIGS. 3B-D, first panel).

In contrast, both the proportion and function of bladder infiltrating NK and CD8⁺ T cells were significantly increased in VSVd51-mGM-CSF treated mice (FIG. 3E, first panel). In addition to immune effector cells, the proportion of regulatory cells was assessed, specifically in the tumor microenvironment of the dissociated bladder. It was not observed any differences in T regulatory cell proportions across treatment groups (FIG. 3F). However, a significant decrease in granulocytic myeloid derived suppressor cells (gMDSC, CD11b⁺/Ly6G^{high}) was observed in VSVd51-treated groups compared to controls, while mice treated with VSVd51-mGM-CSF had reduced gMDSC proportions compared to PBS control, but had a higher proportion compared to the parental virus (FIG. 3G). In contrast, no differences were observed between VSVd51- and VSVd51-mGM-CSF-treated groups for monocytic MDSC (mMDSC) populations other than a general decrease compared to untreated mice (FIG. 3G). Lastly, the functional suppressive capacity of gMDSC populations was examined by gating on arginase 1⁺ (ARG1⁺) gMDSCs and detected a greater proportion in VSVd51-mGM-CSF treated mice compared to VSVd51 (FIG. 3G). Taken together, these results suggest that while VSVd51 treatment diminished both myeloid regulatory cell population levels, treatment with VSVd51-mGM-CSF conversely enhanced the population of ARG1⁺ gMDSCs.

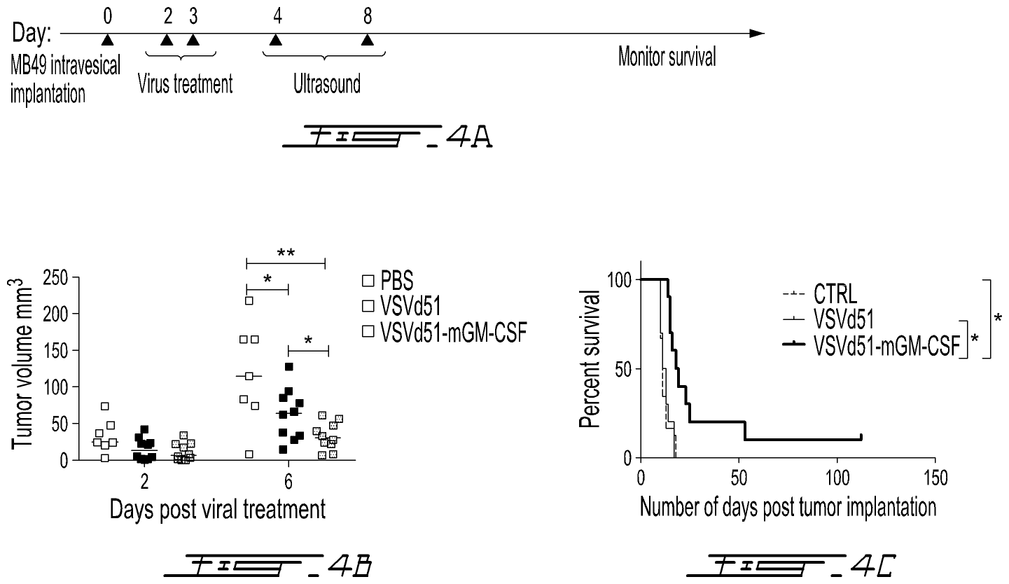

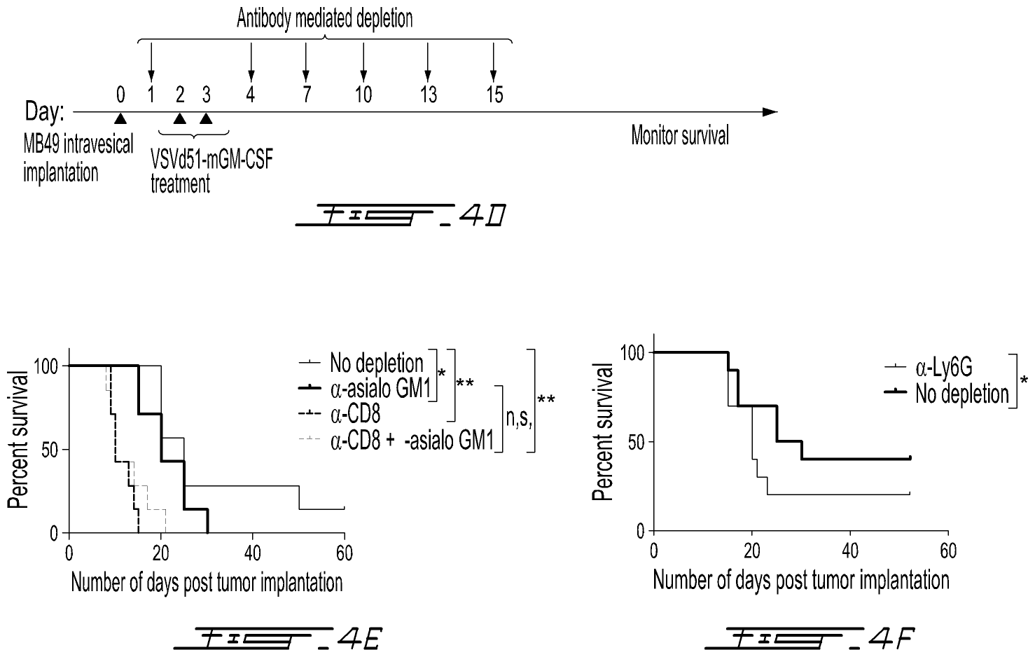

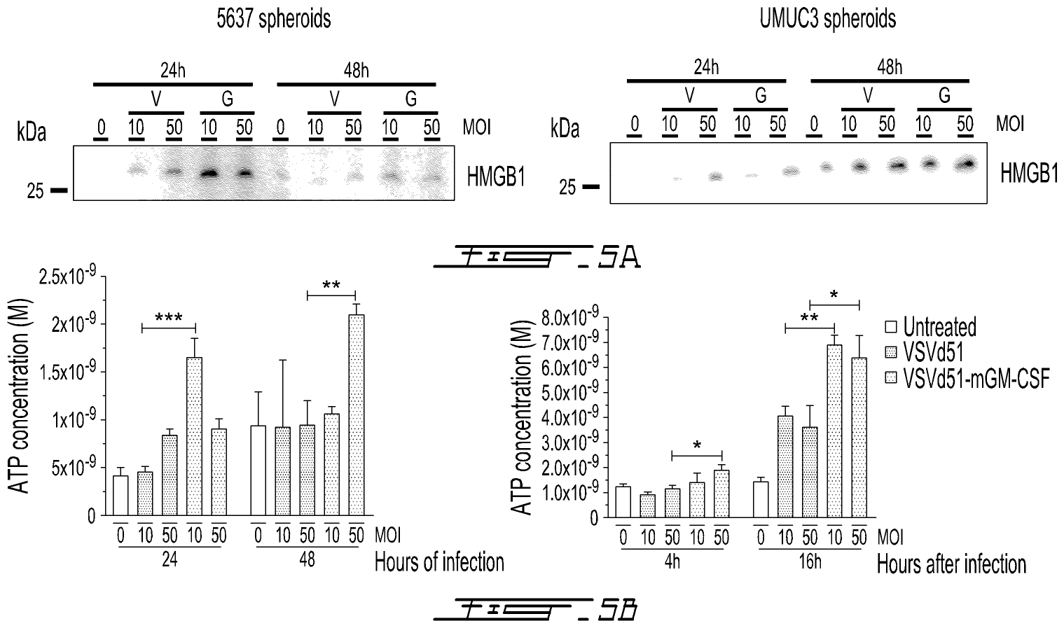

To further investigate whether the improved immune function of treated mice will result in better disease outcome, mice were monitored for survival and measured tumor volume by small animal ultrasound. Reduced tumor volume and improved survival was observed in VSVd51-mGM-CSF-treated mice compared to controls (FIGS. 4A-C). To confirm the critical role of NK and CD8⁺ T cells after virus administration, survival was monitored in VSVd51-mGM-CSF-treated mice that were pharmacologically depleted singly or of both immune cell populations (FIG. 4D). In support of the in vivo data showing enhanced NK and CD8⁺ T cell function (FIG. 3), the protective effect of treated mice with VSVd51-mGM-CSF was partially removed upon depletion of NK cells, but completely abrogated upon depletion of CD8⁺ T cells or combination of NK and CD8⁺ T cells (FIG. 4E). These results suggest that the therapeutic benefit of VSVd51-mGM-CSF treatment is dependent upon both NK and CD8⁺ T cells, but likely more dependent on CD8⁺ T cells. Following pharmacological depletion with anti-Ly6G, a modest, but significant improvement was observed in survival in VSVd51-mGM-CSF treated mice (FIG. 4F). These data suggest that improved survival of mice following VSVd51-mGM-CSF treatment is positively associated with effector immune cells, but negatively associated with myeloid regulatory cells.

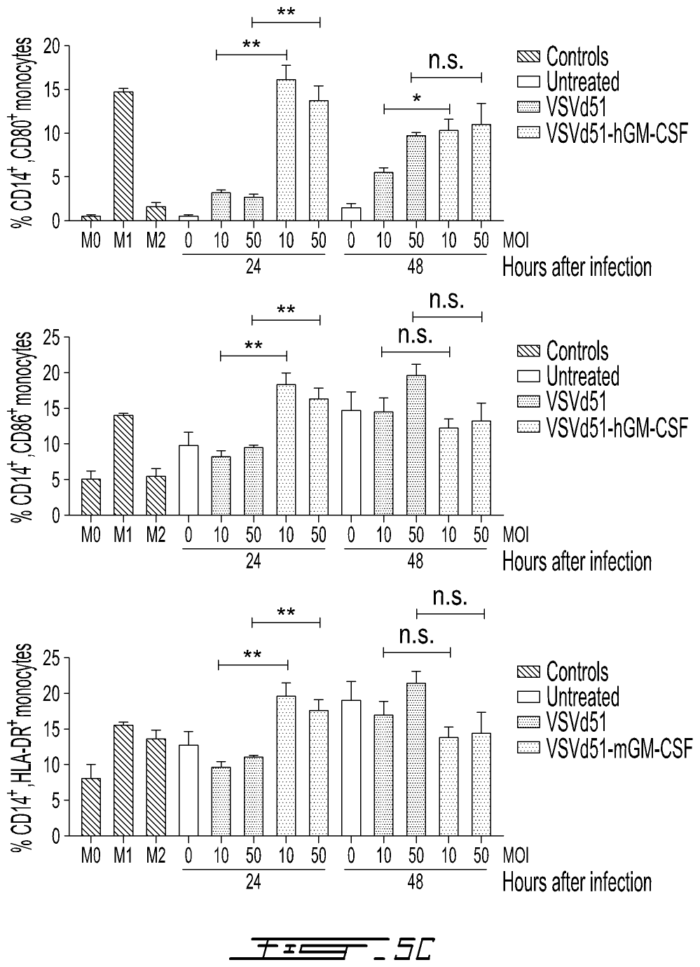

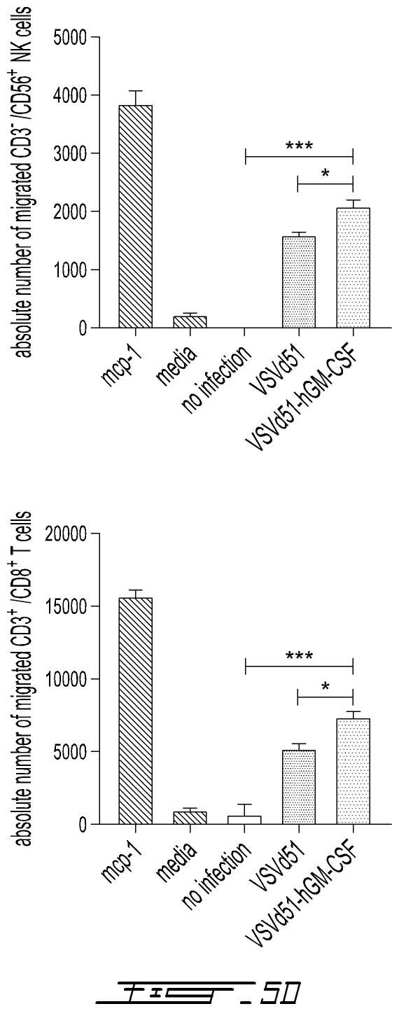

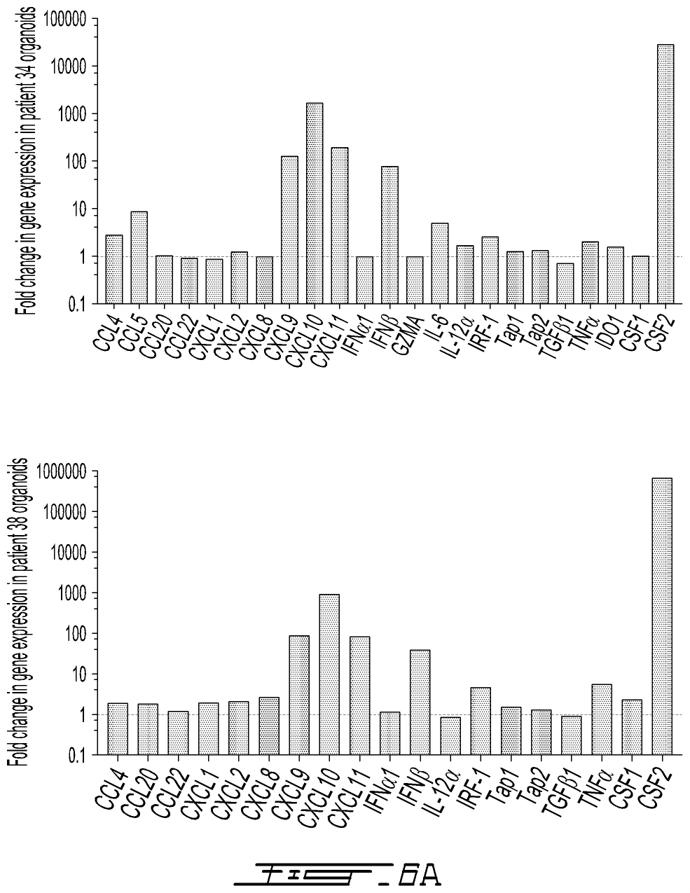

To test the translational potential of VSVd51-hGM-CSF, its anti-cancer effect was examined on human BC cell lines and primary human immune cells. To do so, the human 5637 and UM-UC-3 BC cell lines was propagated as 3D spheroids instead of 2D monolayers to better mimic the physiology of the bladder urothelium. Using these spheroids, biomarkers of ICD were examined including secreted HMGB1 and ATP following infection. In the cell-free supernatants of VSVd51-hGM-CSF infected 5637 spheroids, increased levels of HMGB1 were observed, while similar HMGB1 levels were observed in UM-UC-3 spheroids (FIG. 5A). Increased ATP levels (FIG. 5B) at both 24 and 48 h post-infection compared to VSVd51 and non-infected controls was detected in 5637 spheroids, while higher ATP levels in UM-UC-3 spheroids were detected at earlier time points (4 h, 16 h) due to the stability of the spheroid structures in culture. The presence of these DAMPs suggest a greater induction of ICD in human BC spheroids by VSVd51-hGM-CSF.

In co-culture experiments with CD14+ human monocytes incubated with cell-free lysates from infected 5637 spheroids, polarization of these monocytes was observed towards an M1-like phenotype expressing higher levels of CD80, CD86, HLA-DR and PD-L1 that have been previously suggested to promote anti-tumor immune responses (FIG. 5C). To examine the immune consequences of VSVd51-hGM-CSF infection on immune effector cells, human NK and CD8⁺ T cell migration was measured. It is reported increased migration of CD3⁻/CD56⁺ NK and CD3⁺/CD8⁺ T cells towards CM from infected 5637 spheroids following VSVd51-hGM-CSF infection (FIG. 5D). Taken together, these data using human primary immune cells and human BC spheroids demonstrate the immune activating potential of VSVd51-hGM-CSF.

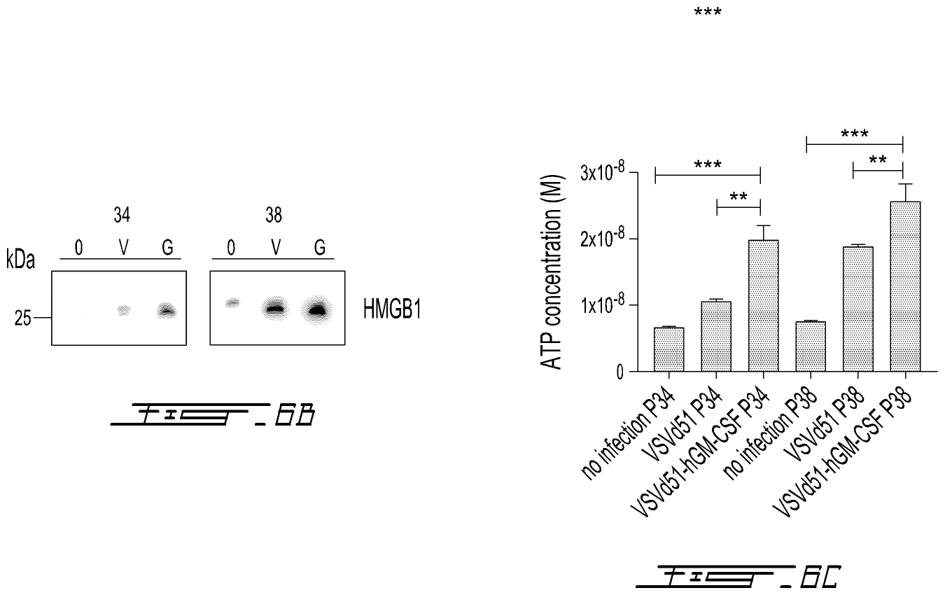

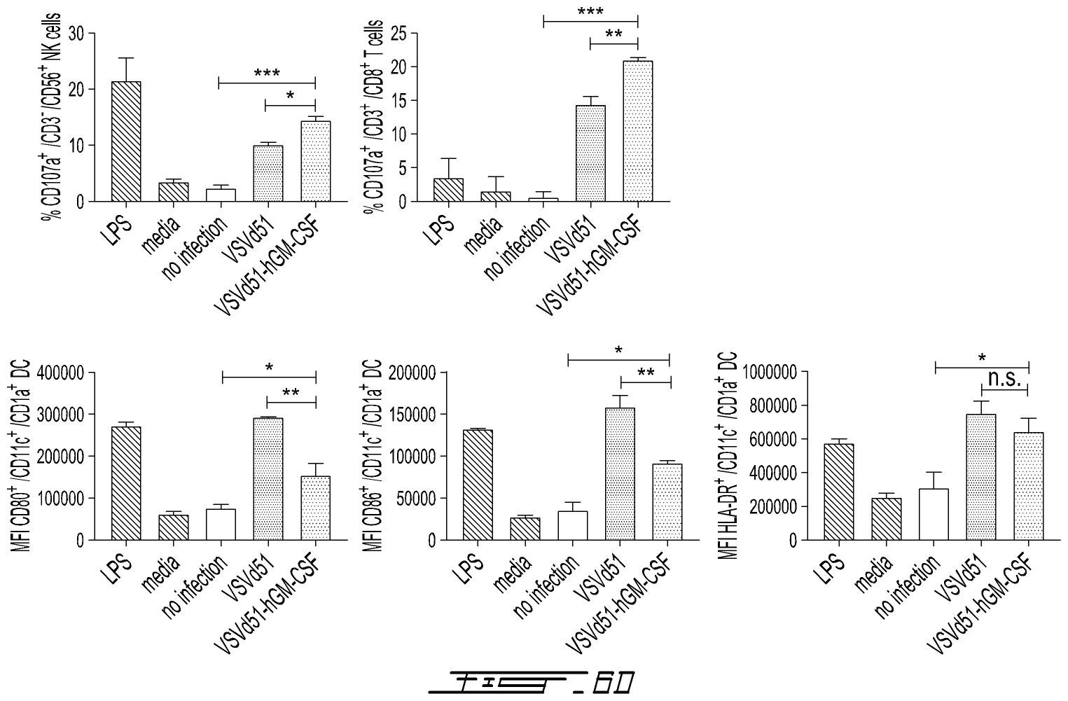

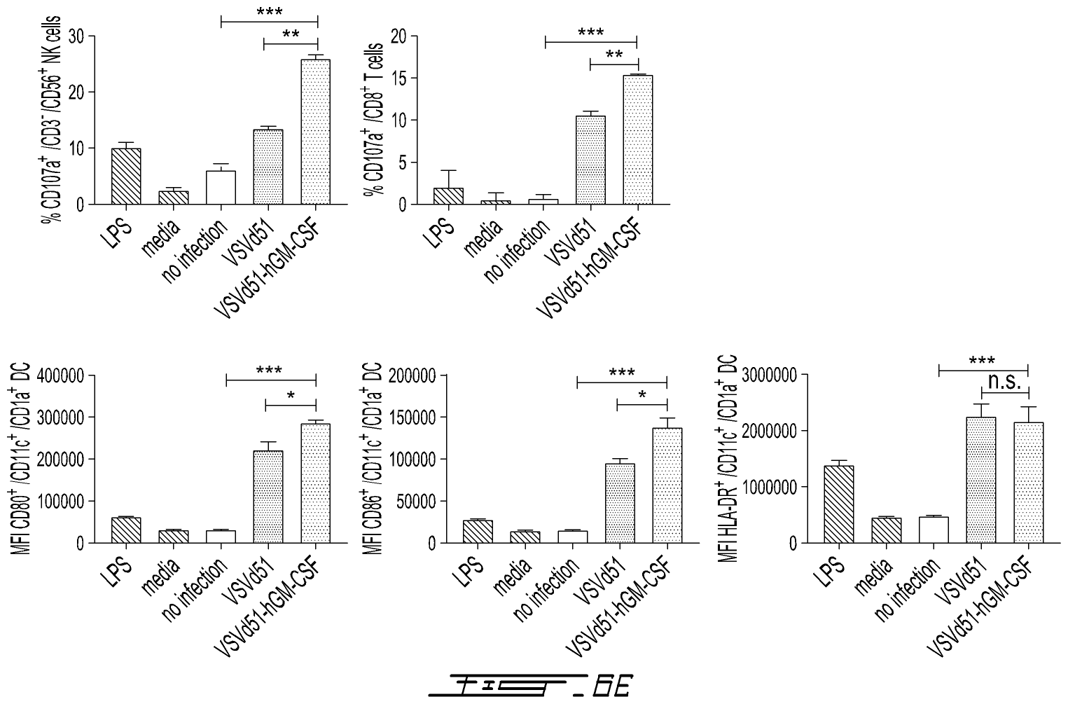

BC patients were enrolled in the observational oVSV-bladder study (Ethics protocol #2018-2414). To better model the physiological structures of the bladder urothelium, these BC patient were propagated derived tissue as 3D organoids ex vivo. Organoids from patient 34 and 38 were infected with VSVd51-hGM-CSF for 6 h and qPCR for gene expression analysis and assays to measure biomarkers of ICD were conducted. Patient 34 displayed an immunogenic gene expression pattern with enhanced expression of multiple immune genes, notably CCL4, CCL5, CXCL9, CXCL10, CXCL11, IFNγ, IL6, IRF-1 and CSF-2 compared to untreated controls. In patient 38—CXCL9, CXCL10, CXCL11, IFNγ, IRF-1, TNFα and CSF-2 gene expression were also increased (FIG. 6A), while genes related to antigen presentation TAP1 and TAP2 were also moderately increased following infection.

Moving beyond immune gene signatures, biomarkers of ICD were compared in VSVd51 infected vs. VSVd51-hGM-CSF infected BC organoids. Biomarkers of ICD including HMGB1 release for both patients was detected at higher levels in VSVd51-hGM-CSF infected organoids compared to VSVd51 infected and uninfected controls (FIG. 6B), while ATP release was detected at higher levels in VSVd51-hGM-CSF infected organoids for patient 34 (FIG. 6C). Importantly, autologous immune cell activation was looked at following treatment of matched BC organoids with VSVd51-hGM-CSF (FIGS. 6D and E). It is noted enhanced CD107a degranulation in NK and CD8⁺T cells following co-culture with autologous CD11c⁺ DC incubated with CM from VSVd51-hGM-CSF infected organoids compared to treatment with VSVd51 and uninfected controls in both patients. Additionally, enhanced CD80, CD86 and HLA-DR expression was detected on CD11c⁺ autologous cDCs of patient 38 following incubation with CM from VSVd51-hGM-CSF treated organoids compared to controls (FIG. 6E). These human data demonstrate that an ICD gene signature is present in patient BC organoids following VSVd51-hGM-CSF infection and this phenotype has the capacity to activate autologous immune cells ex vivo. Taken together, the translational data highlight the clinical potential of using VSVd51-hGM-CSF as a novel immunotherapy to treat BC patients.

Accordingly, from in vitro experiments, it was determined that infection of mouse and human BC cells with VSVd51-mGM-CSF and VSVd51-hGM-CSF, respectively, results in higher ICD compared to VSVd51 infected and non-infected cells. Enhanced release of intracellular HMGB1, ATP and increased Calreticulin⁺/DAPI⁺ tumor cell populations (FIGS. 2 and 5) was observed. Further, an increased pro-inflammatory gene signature was detected in VSVd51-hGM-CSF infected BC patient derived organoids suggesting the generation of anti-tumor immunity. This included numerous genes associated with multiple aspects of immune cell function, including antigen presentation, innate immunity, and T cell activation and recruitment. From in vivo experiments, treatment of MB49 tumor bearing mice with 2 doses of VSVd51-mGM-CSF significantly augmented both systemic and tumor infiltrating innate and adaptive immune cell functionality over VSVd51 resulting in reduced bladder tumor burden and improved survival. Both NK and CD8⁺ T cells are important in contributing towards VSVd51-mGM-CSF mediated treatment efficacy as shown in depletion studies. However, CD8⁺ T cells appear to play a more important role in mediating therapeutic efficacy as evidenced by shortened survival in CD8⁺ T cell depleted mice, singly or in combination with NK cells. This combination of ICD combined with augmented innate and adaptive immune functions effectively creates an in situ vaccination effect.

Interestingly, while the overall survival in VSVd51-mGM-CSF treated mice was improved, there was a proportion of mice who developed large bladder tumors and succumbed to disease faster than the VSVd51 treated group. Upon closer examination, accumulation of ARG1⁺ gMDSC was observed in the bladder tumor tissue of VSVd51-mGM-CSF treated mice (FIG. 3G). These regulatory cells have been shown to expand in the tumor microenvironment and exert suppression on innate and adaptive immunity through production of regulatory enzymes. While enhancing cDC proliferation and activation, GM-CSF may indiscriminately increase myeloid regulatory cell populations. Therefore, the counter-regulatory activities of GM-CSF overexpression by VSVd51-mGM-CSF may be reducing its therapeutic efficacy by expanding MDSC and subsequent inhibition of effector immune cells. Depletion studies with anti-Ly6G in the context of VSVd51-mGM-CSF treatment significantly improved survival in these cohorts (FIG. 4F). While VSVd51-mGM-CSF significantly improves survival compared to VSVd51, MDSC along with other regulatory mechanisms in the tumor microenvironment may be contributing towards suboptimal VSVd51-mGM-CSF efficacy. The counter regulatory activity of GM-CSF is an important limitation that could reduce the therapeutic efficacy of other OV's expressing GM-CSF, such as the FDA approved oncolytic HSV for the treatment of end-stage melanoma. Therefore, combination treatment with OV and immunomodulators to remove tumor microenvironment immune suppression, including myeloid regulatory cells may improve overall survival.

In translational studies, an analogous mechanism of VSVd51-hGM-CSF-induced immune activation is occurring in human BC spheroids and patient organoids. VSVd51-hGM-CSF infection of human BC spheroids resulted in the enhanced release of immunogenic DAMPs, polarization of human monocytes towards an M1-like phenotype and lead to greater NK and CD8⁺ T cell migration (FIG. 5). In 2 BC patient derived organoids, gene expression data following VSVd51-hGM-CSF infection revealed an immunogenic gene signature, while evaluation of ICD biomarkers showed augmented release of DAMPs. Importantly, autologous patient immune cells incubated with CM from VSVd51-hGM-CSF infected matched organoids demonstrated enhanced innate and adaptive immune cell activation compared to VSVd51 or uninfected controls. Taken together, these translational results demonstrate the feasibility of developing a VSVd51-hGM-CSF based immunogenic virotherapy to treat BC. Thus it is provided the use of VSVd51-hGM-CSF in the clinical setting of BCG salvage/bladder sparing therapy to improve survival and promote quality of life in BC patients.

It is therefore provided a composition comprising a VSVd51-hGM-CSF construct as depicted in SEQ ID NO: 1 and a carrier.

The composition provided herewith can be administered by any means, such as e.g. by a route of administration selected from parenteral, intravenous, oral, subcutaneous, intra-arterial, intracranial, intrathecal, intraperitoneal, topical, intranasal and intramuscular.

In an embodiment, the composition described herein could be administered with an immunomodulator (e.g. type I interferon, more preferably interferon alpha (IFNα)) and/or Bacillus Calmette-Guérin (BCG) to said patient. It is encompassed that the composition described herein can be used to treat solid cancer, such as e.g. bladder cancer, pancreatic cancer, breast cancer, colorectal cancer, ovarian cancer, and melanoma.

The terms “functional equivalents” and “functional variants” are used interchangeably herein and such functional equivalents of VSVd51-hGM-CSF are encompassed herein. Functional nucleic acid equivalents may typically contain silent mutations or mutations that do not alter the biological function.

As defined herein, the term “substantially homologous” refers to a first nucleotide sequence which contains a sufficient or minimum number of identical or equivalent nucleotides to a second nucleotide sequence such that the first and the second nucleotide sequences have a common domain. For example, nucleotide sequences which contain a common domain having about 60%, preferably 65%, more preferably 70%, even more preferably 75%, 80%, 85%, 90%, 95%, 96%, 97%, 98% or 99% identity or more are defined herein as sufficiently identical.

As used herein, “pharmaceutical composition” means therapeutically effective amounts (dose) of an agent together with pharmaceutically acceptable diluents, preservatives, solubilizers, emulsifiers, adjuvants and/or carriers. A “therapeutically effective amount” as used herein refers to that amount which provides a therapeutic effect for a given condition and administration regimen. Such compositions may be liquids or lyophilized or otherwise dried formulations and include diluents of various buffer content (e.g., Tris-HCl, acetate, phosphate), pH and ionic strength, additives such as albumin or gelatin to prevent absorption to surfaces, and detergents (e.g., Tween™ 20, Tween™ 80, Pluronic® F68, bile acid salts). Compositions of the invention may also comprise solubilizing agents (e.g., glycerol, polyethylene glycerol), anti-oxidants (e.g., ascorbic acid, sodium metabisulfite), preservatives (e.g., thimerosal, benzyl alcohol, parabens), and bulking substances or tonicity modifiers (e.g., lactose, mannitol) which contribute to covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance of the active agent. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention include particulate forms, protective coatings, protease inhibitors or permeation enhancers for various routes of administration, including parenteral, pulmonary, nasal and oral routes.

In addition, the term “pharmaceutically effective amount” or “therapeutically effective amount” refers to an amount (dose) effective for treating a patient, having, for example, a nerve injury. It is also to be understood herein that a “pharmaceutically effective amount” may be interpreted as an amount giving a desired therapeutic effect, either taken in one dose or in any dosage or route or taken alone or in combination with other therapeutic agents.

A therapeutically effective amount or dosage of an active agent may range from about 0.001 to 30 mg/kg body weight, with other ranges of the invention including about 0.01 to 25 mg/kg body weight, about 0.025 to 10 mg/kg body weight, about 0.3 to 20 mg/kg body weight, about 0.1 to 20 mg/kg body weight, about 1 to 10 mg/kg body weight, 2 to 9 mg/kg body weight, 3 to 8 mg/kg body weight, 4 to 7 mg/kg body weight, 5 to 6 mg/kg body weight, and 20 to 50 mg/kg body weight. In other embodiments, a therapeutically effective amount or dosage of an active agent may range from about 0.001 to 50 mg total, with other ranges of the invention including about 0.01 to 10 mg, about 0.3 to 3 mg, about 3 to 10 mg, about 6 mg, about 9 mg, about 10 to 20 mg, about 20-30 mg, about 30 to 40 mg, and about 40 to 50 mg.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an active compound can include a single treatment or a series of treatments. In one example, a subject is treated with an active compound in the range of between about 0.3 to 10 mg, one time per week for between about 1 to 10 weeks, alternatively between 2 to 8 weeks, between about 3 to 7 weeks, or for about 4, 5, or 6 weeks. It will also be appreciated that the effective dosage of an active compound used for treatment may increase or decrease over the course of a particular treatment.

In an embodiment, the composition described herein is for treating a solid cancer.

In addition to the provided results herein, as seen in Table 1, the composition described herein was used in known cancer cell lines:

TABLE 1
Anti cancer effect measured in multiple cell
lines of the VSVd51-hGM-CSF construct.

| | ATP | HMGB1 | Calreticulin | |
|---|---|---|---|---|
| | biomarker of | biomarker of | biomarker of | |
| human | immunogenic | immunogenic | immunogenic | |
| cell lines | cell death | cell death | cell death | comments |
| Panc1 | yes | yes | | Pancreatic cancer cell line |
| Miapaca2 | Yes | Yes | | Pancreatic cancer cell line |
| UMUC3 | yes | yes | yes | Bladder cancer cell line |
| 5637 | yes | yes | yes | Bladder cancer cell line |
| T24 | Yes | yes | | Bladder cancer cell line |
| TCCUSP | yes | yes | | Bladder cancer cell line |

In another embodiment, the solid cancer is bladder cancer, pancreatic cancer, breast cancer, colorectal cancer, ovarian cancer, or melanoma.

### Example I

### VSVd51-hGM-CSF Characterization

MB49 were maintained in DMEM; 5637 in RPMI; UM-UC-3 in EMEM, all supplemented with 10% HI FBS+100 U/ml penicillin and 100 μg/ml streptomycin (complete media). 5637 and UM-UC-3 cell lines were purchased from ATCC and MB49 cell line from Millipore-Sigma and were verified to be mycoplasma free and show appropriate microscopic morphology. VSVd51 expressing human GM-CSF (VSV-hGM-CSF) was cloned from parental VSVd51 expressing GFP (VSVd51). Briefly, polymerase chain amplification was used to amplify hGM-CSF from the pUNO1-hGM-CSF plasmid (Invivogen) and the cDNA was subcloned into VSVd51 via the restriction sites XhoI/NheI. This plasmid was used to rescue a recombinant VSVd51-hGM-CSF as previously described (Alkayyal et al., 2017, Cancer Immunol Res, 5: 211-222). VSVd51 and VSVd51-mGM-CSF were obtained from the Ottawa Hospital Research Institute (Ottawa, Canada). All viruses were propagated on Vero cells and purified using Opti-Prep purification methods. Viral titers were determined by a standard plaque assay as previously published (Alkayyal et al., 2017, Cancer Immunol Res, 5: 211-222). Viral cytotoxicity was assessed on the indicated cell lines, and cell viability was carried out as described previously (Alkayyal et al., 2017, Cancer Immunol Res, 5: 211-222).

Female C57BI/6 mice (6-8 weeks old, 20-25 g) were purchased from Charles River (Quebec). Animals were housed in pathogen-free conditions at the Central Animal Facility of the Universite de Sherbrooke with access to food/water ad libitum. Animals were euthanized by cervical dislocation under anesthesia. All studies were conducted in accordance with university guidelines and the Canadian Council on Animal Care and protocols were approved by the Faculty of Medicine and Health Sciences Animal Care Committee.

For orthotopic implantation of BC cells, mice were anaesthetized and chemical lesions were induced by intravesical instillation of trypsin (Wisent) 1:1 in DMEM. During this procedure, all mice were kept under anesthesia (3% induction, 1.5% maintenance of isoflurane with 2% O₂). Subsequently, 5×10⁵ MB49 bladder tumor cells were instilled. Two days later, each group of mice received via intravesical instillation of 50 μl of 5×10⁸ PFU of VSVd51 or VSVd51-mGM-CSF or vehicle for control groups. For the in vivo depletion of immune cell populations, 6 doses of depletion antibodies (1 dose 24 hours after tumor instillation, followed by 5 additional doses 3 days apart) were administered by intraperitoneal injection of 250 μg/dose for anti-mouse Ly6G (1A8; BioXCell); 20 μg/dose for anti-Asialo (GM1, Life Technologies) and 250 μg/dose for anti-CD8a (53-6.7, BioXCell). Bladder tumor growth was monitored bi-weekly by small animal ultrasound (Vevo3100, VisualSonics).

Following euthanasia, bladders were immediately placed in cRPMI and processed fresh using the mouse tumor dissociation kit (Miltenyi biotec). Briefly, tumors were cut into small pieces (<2 mm 3), then treated with dissociation enzymes and placed into the gentle MACS OctoDissociator (Mitenyi biotec). Following dissociation, macroscopic pieces were removed using a 70 μm nylon cell strainer. Single cell suspensions were washed twice in cRPMI and proceeded to flow cytometry acquisition and analysis as described below.

To analyze spleen, blood and tumor dissociated lymphocyte populations, an initial incubation was done in ACK lysis buffer for 5 mins to lyse red blood cells. 1×10⁶ cells were then added to each tube. Fc block was added prior to antibody staining for 10 mins at room temperature. Samples were washed twice with flow cytometry buffer (PBS, 2% FBS, 1 mM EDTA) and acquired on a CytoFLEX 30 (Beckman Coulter). Data was analyzed with CytExpert software. For assessment of NK and T cell functionality, cells were cultured with PMA/ionomycin (Sigma) for 4 h in the presence of brefeldin A (1 μl/ml) at 37° C. After 4 h, cells were washed twice with PBS, and then stained for NK and T cell markers. Cells were then fixed and permeabilized using BD Cytofix/Cytoperm kit, according to the manufacturer's protocol, and intracellular staining for granzyme B and IFNγ was performed. The CD107a antibody was added to cells alongside PMA/ionomycin stimulation.

For monolayer cultures, conditioned media (CM) was obtained by seeding 5×10⁵ cells in 12-well plates in their corresponding media for 24 h followed by infection with VSVd51 and VSVd51-m/hGM-CSF at the indicated PFU for the indicated time points. For 5637 spheroids, CM was obtained by resuspending 2.5×10⁴ 5637 cells in 20 μl of Matrigel (Corning) per well of a 48-well (Thermo Fisher Scientific) plate for 6 days followed by infection with VSVd51 and VSVd51-hGM-CSF. Infected cells were harvested and processed as described above. Bioimaging was performed using an inverted microscope (Zeiss). Western blot: HMGB1 protein from CM was resolved by SDS-PAGE and transferred to Immun-Blot-PVDF membranes (BioRad) for immunoblotting. Protein expression was detected using HMGB1 primary antibodies (1:1000) and corresponding HRP-conjugated secondary antibodies (1:10000). Protein expression was visualized by chemiluminescence detection (Azure 600, Azure Biosystems). For Adenosine 5′-triphosphate (ATP) detection, the concentration of ATP in the CM was measured with the ENLITEN-ATP kit (Promega). Briefly, 100 μl of CM were transferred to 96-well opaque plates. 100 μl of reconstituted rLuciferase/Luciferin reagent was added to each well followed by measurement of luciferase activity using a luminescence microplate reader (Fusion V3.0).

Total RNA was extracted from virus-infected or mock-infected cells or organoids using Trizol (Invitrogen) according to the manufacturer's protocol. RNA was then used for reverse transcription and qPCR which was performed by the RNomics Platform at the Universite de Sherbrooke. RNA integrity was assessed with an Agilent 2100 Bioanalyzer (Agilent Technologies). Reverse transcription was performed on 1.1 μg total RNA with Transcriptor reverse transcriptase, random hexamers, dNTPs (Roche Diagnostics), and 10 units of RNAse OUT (Invitrogen) following the manufacturer's protocol in a total volume of 10 μl. All forward and reverse primers (Supp. Table S2) were individually resuspended to 20-100 μM in Tris-EDTA buffer (IDT) and diluted as a primer pair to 1 μM in RNase DNase-free water (IDT). The amplified products were analyzed by automated chip-based microcapillary electrophoresis on Labchip GX Touch HT instruments (Perkin Elmer). QPCR reactions were performed in 10 μl in 384 well plates on a CFX-384 thermocycler (BioRad) with 5 μl of 2× PerfeCTa® SYBR® Green Supermix (Quantabio), 10 ng (3 μl) cDNA, and 200 nM final (2 μl) primer pair solutions. The following cycling conditions were used: 3 min at 95° C.; 50 cycles: 15 sec at 95° C., 30 sec at 60° C., 30 sec at 72° C. Relative expression levels were calculated using the qBASE framework and the housekeeping genes RMRP, RNU6-4P and rRNA 5.8s for human cDNA. For every qPCR run, control reactions performed in the absence of template were performed for each primer pair and these were consistently negative. Amplicon sizing and relative quantitation were performed by the manufacturer's software.

Culture supernatants were diluted 5-fold. ELISA kits (Peprotech) for detecting mouse and human GM-CSF were performed according to manufacturer's instructions.

Human monocytes were isolated from peripheral blood (Human CD14⁺ isolation kit, Stemcell). 5×10⁵ monocytes were seeded in 24-well plates in complete RPMI and incubated overnight at 37° C. and 5% CO 2. 24 h later, the monocyte media was replaced with the CM of infected human cell lines. For controls, monocytes were co-cultured with recombinant human IL-10, IL-4, and TGFβ (BioBasic Inc) all at a final concentration of 20 ng/ml for differentiation to M2-like macrophages; and with LPS (50 ng/ml) (Millipore Sigma) and recombinant human IFNγ (20 ng/ml) (BioBasic Inc) for M1-like macrophages. Undifferentiated monocytes remained in complete media as M0. Following overnight incubation, cells were harvested and processed for flow cytometry as described above. 200 μl of CM were placed in the lower well of Boyden chambers, separated from the top well by a 5 μm-pore polycarbonate filters (Neuro Probe). 6×10⁵ human PBMC was added to the top chamber, followed by incubation at 37° C., 5% CO₂ for 45 mins. Next, the media in the top of the chamber was aspirated and the membrane removed with forceps. This was followed by harvesting of media in the bottom chamber and quantification of migrated cells by Trypan Blue exclusion. The cells were stained and acquired by flow cytometry as described above.

Bladder tumor tissue from patients 34 and 38 were collected after surgery (Human protocol #: 2018-2465, approved by the ethics board of CIUSSS de l'Estrie CHUS) and placed in cDMEM. Tumors were dissociated using the human tumor dissociation kit (Miltenyi biotec) according to the manufacturer's recommendations. Briefly, tumors were cut into small pieces (<2 mm 3), then treated with dissociation enzymes and placed into the gentle MACSOctoDissociator (Miltenyi biotec). Macroscopic pieces were removed using 70 μm nylon cell strainers. Tumor cells were washed twice in DMEM. Cells were viably frozen down or freshly used for downstream experiments.

Viably frozen or freshly dissociated cells (1×10⁵) were collected by centrifugation and resuspended in 20 μl of matrigel (Corning) and plated in a prewarmed 48-well plate. When the matrigel was solidified, human bladder organoid media was added [Adv. DMEM/F-12, 100 ng/ml FGF10, 25 ng/ml FGF7, 12.5 ng/ml FGF2 (Peprotech), 1×B27 supplement (ThermoFisher), 5 μM A83-01, 1.25 mM N-acetylcysteine, and 10 mM nicotinamide (sigma)]. Human BC organoids were passaged biweekly by dissociation using TrypLE (ThermoFisher). 10 μM ROCK inhibitor (Y-27632) was added to the media after passaging to prevent cell death. Organoids were frozen in freezing media (90% FBS, 10% DMSO) and could be recovered efficiently. OV infection of organoids were performed as described for 5637 and UM-UC-3 spheroids in immunogenic cell death assays.

Immature DCs were obtained by CD14 positive selection (StemCell) according to manufacturer's guidelines from frozen human PBMCs. Sorted cells were incubated for 6 days with 500 U/ml of recombinant human IL-4 and 50 ng/ml of recombinant human GM-CSF (Bio Basic). For DC:PBMC co-culture assays, matched PBMCs were thawed incubated for 24 h with 100 U/ml of recombinant human IL-2 (Bio Basic). Following this, both DCs and lymphoid cells were incubated an additional 24 h with CM from infected autologous organoids and acquired by flow cytometry. T and NK cell functionality were assessed as described above.

All statistical analyses were conducted using Prism 7 (GraphPad). Unpaired two-tailed t tests were used for comparing uninfected or infected cells or differentially treated mice. Survival differences of tumor-bearing and treated mice were assessed using Kaplan-Meier curves and analyzed by log-rank testing. P<0.05 was considered as statistically significant.

While the description has been described in connection with specific embodiments thereof, it will be understood that it is capable of further modifications and this application is intended to cover any variations, uses, or adaptations and including such departures from the present disclosure as come within known or customary practice within the art to and as may be applied to the essential features hereinbefore set forth, and as follows in the scope of the appended claims.

1. A composition comprising:
(a) (i) VSVd51-hGM-CSF construct as depicted in SEQ ID NO: 1,
(ii) or a functional equivalent thereof, or
(iii) a nucleotide sequence having at least 70% identity with SEQ ID NO: 1; and
(b) a carrier.

2. The composition of claim 1, for treating a solid cancer.

3. The composition of claim 2, wherein the solid cancer is bladder cancer, pancreatic cancer, breast cancer, colorectal cancer, ovarian cancer, or melanoma.

4. The composition of claim 1, formulated for an administration selected from parenteral, intravenous, oral, subcutaneous, intra-arterial, intracranial, intrathecal, intraperitoneal, topical, intranasal and intramuscular.

5. A method of treating a solid cancer comprising administering to a patient in need thereof a composition comprising a vesicular stomatitis virus (VSV) and a growth factor.

6. The method of claim 5, wherein the VSV is VSVd51.

7. The method of claim 5, wherein the growth factor is a human growth factor.

8. The method of claim 7, wherein the human growth factor is granulocyte macrophage-colony stimulating factor (GM-CSF).

9. The method of claim 5, wherein the composition comprises VSVd51-hGM-CSF construct as depicted in SEQ ID NO: 1.

10. The method of claim 5, further comprising administering an immunomodulator to said patient.

11. The method of claim 10, wherein the immunomodulator is a type I interferon.

12. The method of claim 11, wherein the immunomodulator interferon alpha (IFNα).

13. The method of claim 1, further comprising administering Bacillus Calmette-Guérin (BCG) to said patient.

14. The method of claim 1, wherein the solid cancer is bladder cancer, pancreatic cancer, breast cancer, colorectal cancer, ovarian cancer, or melanoma.

15. The method of claim 1, wherein the solid cancer is bladder cancer.

16. The method of claim 1, wherein said composition is administered parenterally, intravenously, orally, subcutaneously, intra-arterially, intracranially, intrathecally, intraperitoneally, topically, intranasally and intramuscularly.

17. The method of claim 1, wherein said composition increases expression of at least one of CD80, CD86, HLA-DR and PD-L1 in said patient.

18. The method of claim 1, wherein said composition increases ATP levels in said patient.

19. The method of claim 1, wherein said composition increases gene expression of at least one of CCL4, CCL5, CXCL9, CXCL10, CXCL11, IFNγ, IL6, IRF-1, CSF-2, TNFα, CSF-2, TAP1 and TAP2 compared to untreated controls.

20-33. (canceled)

34. A method of treating a solid cancer comprising administering to a patient in need thereof the composition of claim 1.


SEQUENCE LISTING (1 sequence)

SEQ ID NO: 1 - DNA, 10457 bp, human
tgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgctaggtatgaaaaaaactaacagatatcacgctcgagatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagctagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc